(19) United States
(12) Patent Application Publication
(10) Pub. No.: US 2012/0210468 A1
(43) Pub. Date: Aug. 16, 2012

(54) Novel method to generate commercially useful oils in algae

(75) Inventors: Chifu Huang, Lexington, KY (US); Jeffrey Lee Ebersole, Lexington, KY (US)

(73) Assignee: Dr. Chifu Huang, Lexington, KY (US)

(21) Appl. No.: 13/029,115

(22) Filed: Feb. 16, 2011

(51) Int. Cl. C12N 15/74; C12N 15/82

(52) U.S. Cl. 800/294; 435/471

(57) ABSTRACT

The present invention discloses a novel microalgae-based method, called Microalgae Genomics Technology™ (MaGT), which can be used to generate and improve oil/fatty acid production in microalgae, and revealed some examples of its potential applications. Specifically, this method utilizes microalgae genomics technology to manipulate the intrinsic oil/fatty acid metabolic pathways in the genomes of microalgae and therefore induce the algae to produce novel oils/fatty acids or increase the concentration of existing oils/fatty acids with a variety of commercial applications.

### FIELD OF THE INVENTION

Algae-based natural products are widely used as potential novel pharmaceuticals, nutraceuticals, cosmetics, or even as food. Even though algae possess a broad phytochemical capacity, algae do not necessarily always reveal their functional potential due to certain environmental constraints. Genomics technology has been successfully used in plant and agricultural crops to produce novel traits in plants, such as disease-resistance or insect-resistance in commercial plants. However, genomics technology has seldom been applied to algae. In this invention, we have developed a novel technology, Microalgae Genomics Technology™ (MaGT), which could produce recombinant algae that possess new traits and produce novel oil/fatty acids with commercial applications.

### BACKGROUND OF THE INVENTION

Algae, especially microalgae, are a group of organisms that has received a great deal of attention during the push for “green energy” and help manage global climate change. These organisms have many advantages, including low cost to produce and resulting large biomass. On a per acre scale, algae can produce as many as 30 times more biomass than other crops, such as corn. Most algae can live in simple condition such as H₂O, a carbon source, and sunlight as algae can perform photosynthesis to produce energy for their metabolism. Thus, algae could become a strong candidate as a potential renewable energy source for mankind, since their fatty acids can be extracted and have been suggested for use as biodiesel fuel. Likewise, algae are also attractive renewable natural resources for useful natural products and bioactive compounds.

Microalgae can be grown in mass in both open-culture systems such as ponds, lakes and raceways, or in highly controlled closed-culture systems, similar to those used in commercial fermentation processes, such as for E. coli and mammalian cells. Certain microalgae are very suitable for open system culture where the environmental conditions are very specific, such as high salt or high alkaline ponds, lakes, or lagoons. The extreme nature of this environment severely limits the growth of competitive species, although other types of organisms may still contaminate the culture. The advantage of such systems is that they are generally a low investment, very cost-effective, with high added value, and easy to manage. Closed-culture systems, on the other hand, require significantly higher investments and operating costs, but are independent of all variations in agro-climatic conditions and are very closely controlled for optimal performance and quality. Algae products have substantial potential to be explored for food, production of medicinal products, or as an energy source by man. The repertoire of algae products can be explored in the future to meet human's increasing demand for consumption and need for new products. It is reported that the algae genome shows approximately 120 million nucleotides, and shares nearly 7,000 genes with other organisms, with more than a third of these being shared with both humans and flowering plants. Currently, there is no genomic method available to stimulate or make these algae species produce oils/fatty acids. There is a strong need for such a technology in algae and market for potential novel algal compounds.

A proprietary platform biotechnology, Microalgae Genomics Technology™ (MaGT), has been developed at Oraceuticals, Inc. The basis of MaGT™ is that random activation mutations are induced in a large number of microalgal cells using the intrinsic pathways. These cells then form a population of mutant clones, in which each clone is over-expressing one or more activated genes. Extracts from individual clones are then tested for the required biological activity in a high throughput screen (HTS). With this MaGT technology (see examples) and (high-throuput screening) HTS screening method, we were able to produce novel compounds in the mutant algae that included novel oils/fatty acids, and also increased the production of existing oils/fatty acids.

### SUMMARY OF THE INVENTION

The present invention is to provide a novel method, Microalgae Genomics Technology™ (MaGT), which could enable algae/ or microalgae to produce novel oils/fatty acids or increase the yield of existing oils/fatty acids. Specifically, the Microalgae Genomics Technology™ (MaGT) utilizes random activation mutations which induced in a large number of microalgae cells. These cells then form a population of mutant clones, in which each clone is over-expressing one or more activated genes. Some of these mutants may have activated genes involved in the enzymatic pathways of oils/fatty acids metabolites. These algae mutants may produce novel oils/fatty acids or have increased yields for selected oils/fatty acids. The Microalgae Genomics Technology™ (MaGT) is conveyed as a novel method and a new art to generate oils/fatty acids for biofuels, drug discovery, pharmaceuticals, nutraceuticals, agricultural chemicals, nutritional chemicals, but not limited to.

The characteristics of the above technology are:
(1) The Microalgae Genomics Technology™ (MaGT) utilizes random activation mutations through agrobacteria that induce a large number of mutant microalgal cells. These cells then form a population of mutant clones, in which each clone is over-expressing one or more activated genes. Some of these mutants may have activated genes involved in the enzymatic pathways of secondary metabolites.
(2) MaGT can be used to generate novel oil/fatty acids, which can be screened and the clones can be isolated.
(3) MaGT can be used to generate novel oils/fatty acids or to increase the amount of fatty acids (EPA, DHA, ALA) and omega-6 fatty acid (ARA, GLA), as their fatty acid methyl esters, and fatty acid ethyl esters have anti-microbial activity, including novel anti-microbial activities against oral pathogens.
(4) MaGT can be used to generate novel fatty acids or to increase the amount of fatty acids (EPA, DHA, ALA) and omega-6 fatty acid (ARA, GLA), and their fatty acid methyl esters, and fatty acid ethyl esters. These esters can be used as biofuels.
(5) MaGT can be used as a novel method and a new art to generate oils/fatty acids for biofuels, drug discovery, pharmaceuticals, nutraceuticals, agricultural, nutritional uses, but not limited to.

### DETAILED DESCRIPTION OF THE INVENTION

The following examples are presented by way of illustration, not of limitation.

### Example 1

First, the microalgae species, Chlorella vulgaris, are mutagenized using activation tagging mutagenesis (ATM) by transfer of DNA from Agrobacterium tumefaciens into the algae genome. This technology randomly incorporates viral enhancer sequences together with a bacterial gene for antibiotic resistance into the microalgae genome. The algae cells are then selected for the ability to grow in hygromycin-containing medium, which indicates that the “tag” (T-DNA) construct has been successfully incorporated. The enhancer sequences activate the expression of genes in the immediate vicinity of their site of incorporation and, in the case of C. vulgaris, a population of ˜100,000 mutant cells should “saturate” most of the genome with activation mutations. These cells were then allowed to grow to form ˜100,000 recombinant clones in semi-solidified medium. These clones are screened for oils/fatty acids and isolated by picking up individual clones. In some cultures, the random activation of a gene or genes by ATM should have a specific impact on oils/fatty acids metabolism of those clonal cultures. This process is the key component of our Microalgae Genomics Technology™ (MaGT)

### Example 2

The fatty acids of recombinant C. vulgaris algae clones were extracted with hexane and esterified with ethanol under acidic condition. The fatty acid ethyl esters were analyzed in thin layer chromatography (TLC) for their chemical identities. The TLC data confirmed that some of the clones have significant increases in certain types of fatty acids, such as EPA, as compared to the original wildtype C. vulgaris. Three clones with the highest EPA production were isolated.

### Example 3

The fatty acids of recombinant C. vulgaris algae clones were extracted with hexane and esterified with ethanol under acidic condition. The fatty acid ethyl esters were analyzed in a GC/MS for their chemical identities. The GC/MS data confirmed that some of the clones have significant increases in certain types of fatty acids, such as DHA, as compared to the original wildtype C. vulgaris. Three clones with the highest DHA production were isolated.

### BEST MODE FOR CARRYING OUT THE INVENTION

The preferred embodiments of this invention are described herein, which includes the best mode that is known to the inventor for implementing the invention. Even though the invention has been described with reference to particular embodiments, it is to be understood that these embodiments are merely illustrative of the applications and principles of the invention. Therefore, it is to be understood that many modifications may be made to the illustrative embodiments without departing from the spirit and the scope of the invention as defined in the attached claims.

### INDUSTRIAL APPLICABILITY

This invention is capable of producing novel compounds in algae and microalgae in large scale, and can be used in a variety of applications where oils/fatty acids of algae can be increased. These novel oil/fatty acids could be used as ingredients for consumer goods including foods and beverages, medical devices, medical supplies, dental products, pharmaceuticals, functional foods, drugs, and/or in industrial products, like biofuel.

In addition, this invention technology is also capable of increasing the yields of existing oils/fatty acids in algae and microalgae. These existing oils/fatty acids could be used as ingredients for consumer goods including foods and beverages, medical devices, medical supplies, dental products, pharmaceuticals, functional foods, drugs, and/or in industrial products, like biofuel.

1. The present invention relates to the application of genomics technology to generate novel oils/fatty acids production in microalgae species. Specifically, the invention conveys a novel microalgae-based method, called Microalgae Genomics Technology™ (MaGT), which can manipulate the intrisic oil/fatty acid metabolic pathways in the genome of microalgae species, such as Chlorella vulgaris, to generate novel oils/fatty acids with potential commercial use.

2. The present invention relates to the application of genomics technology to increase selected existing oil/fatty acid production in microalgae species. Specifically, the invention conveys a novel microalgae-based method, called Microalgae Genomics Technology™ (MaGT), which can munipulate the intrinsic oil/fatty acid metabolic pathways in the genome of microalgae species, such as Chlorella vulgaris, to increase selected existing oils/fatty acids with potential commercial use.

3. As mentioned in claim 1, the application of the Microalgae Genomics Technology™ (MaGT) can be used in microalgae, such as Chlorella vulgaris, but not limited to C. vulgaris. It can also apply to other microalgae, such as members of the Spirulina, Scenedesmus, Cyanobacteria, Haematococcus, Crypthecodinium, Schizochytrium, Dunaliella, Odontella, Porphyridium, Shizochytrium, Isochrysis, Aphanizomenon, Rhodophyta genera. For example, Spirulina platensis, Dunaliella salina, Haematococcus pluvialis, Porphyridium cruentum, Scenedesmus almeriensis, Phaeodactylum tricornutum, Dunaliella bardawil, Chlamydomonas nivalis, Porphyridium cruentum, Phaeodactylum tricornutum, Scenedesmus obliquus, Isochrysis galbana, Porphyridium cruentum, Chlorella minutissima, Tetraselmis suecica, Tetraselmis suecica, Nannochloropsis, Isochrysis galbana, Scenedesmus almeriensis, Botryococcus braunii, Arthrospira maxima, Isochrysis galbana, Spirulina maxima, Diacronema vlkianum, Chaetoceros calcitrans, C. gracilis, S. costatum, T pseudonana, Platymonas lutheri, Prymnesiophytes lutheri, Isochrysis sp. and Chroomonas salinaium.

4. The application of the Microalgae Genomics Technology™ (MaGT) can be used in macroalgae and microalgae, but not limited to microalgae. For example, it can apply to macroalgae, such as members of the Halimeda, Caulerpa, Seaweed, Caulerpa, Fucus, Gracilaria, Laminaria, Macrocystis, Monostroma, Porphyra genera.

5. The Microalgae Genomics Technology™ (MaGT) is a novel method that generate oils/fatty acids that can be used for biofuels, drug discovery, pharmaceuticals, nutraceuticals, agricultural chemicals, phytochemicals, and nutritional chemicals, but is not limited to these specified commercial uses.

6. But it is not limited to oils/fatty acids, the Microalgae Genomics Technology™ (MaGT) can also be applied to produce hydrocarbon compounds, such as squalenes, which can be used in biofuels.
